(19) United States
(12) Patent Application Publication
(10) Pub. No.: US 2012/0035595 A1
(43) Pub. Date: Feb. 9, 2012

(54) URETHRAL CATHETER FOR MEASURING THE PRESSURE INSIDE THE BLADDER OF A LIVING BEING

(76) Inventors: Oliver Goedje, Strasslach (DE); Manu Malbrain, Lovenjoel (BE)

(21) Appl. No.: 13/263,817

(22) PCT Filed: Mar. 25, 2010
(86) PCT No.: PCT/EP2010/053883

§ 371 Date: Oct. 10, 2011

(30) Foreign Application Priority Data
Apr. 9, 2009 (DE) 10 2009 017 033.2

(51) Int. Cl. A61M 27/00

(52) U.S. Cl. 604/544

(57) ABSTRACT

A urinary catheter for measuring the pressure in the bladder of a living being comprises a gas-filled measurement balloon for sensing bladder pressure which is arranged on the catheter body distally from a blocking balloon. The catheter body is locally reinforced in the region of the measurement balloon by a reinforcement ring in order to avoid effects of a local deformation of the catheter body on the measurement result. Similarly to conventional Foley catheters, the blocking balloon primarily prevents the urinary catheter from being withdrawn too far from the bladder, which is undesirable. Owing to a suitably selected distance d, which is preferably 3 cm or less, between the measurement balloon and the blocking balloon, the latter is protected from becoming trapped between a fold in the bladder.

The invention relates to a urinary catheter for measuring the pressure in the bladder of a living being.

Many devices for measuring bladder pressure are known from the prior art. A urinary catheter comprising a catheter balloon which serves as a blocking balloon is known from patent U.S. Pat. No. 4,538,621. The pressure is measured via the liquid column of an open lumen. In order to measure internal bladder pressure patent U.S. Pat. No. 6,447,462 B1 describes a balloon catheter comprising a connectable external pressure measuring unit which makes it possible to measure bladder pressure via the internal pressure of the balloon. The gas pressure is transferred from the interior of the balloon to the pressure sensor via a gas-filled balloon lumen.

If the blocking balloon provided to fix the catheter in the bladder simultaneously senses bladder pressure, the pressure values measured are generally corrupted by the contact of the balloon against the wall of the bladder. In principle, the use of measuring balloons carries the risk of faulty measurements owing to the irregular structure of the bladder, which includes folds in the bladder.

By contrast, the measurement of pressure via the liquid column of an open lumen is associated with the drawback that, owing to the hydrostatic pressure of the liquid column, the measured values obtained depend on the geodetic position of the pressure sensor relative to the distal end of the measurement lumen.

Based on the above, the object of the present invention is to provide a device which makes it possible to measure the pressure inside the bladder more reliably than the devices known from the prior art.

The object is solved with a urinary catheter for measuring the pressure in the bladder of a living being, which urinary catheter comprises a catheter body, a balloon lumen connecting a blocking balloon to a proximal port, means attached to the catheter body distally from the blocking balloon in order to sense the pressure to be measured, and a urine drainage lumen connected to at least one distal opening.

In accordance with a preferred embodiment the means for sensing the pressure to be measured comprise a measuring balloon which is connected to a further proximal port via a measurement lumen. The catheter body is preferably reinforced in the region of the measurement balloon in order to minimise the risk of effects of deformation of the catheter body on the measurement result. The measurement lumen may also advantageously be reinforced. The measurement balloon itself and the measurement and evaluation equipment connected proximally to the catheter can basically be provided in a manner identical or similar to that of conventional balloon catheters used to measure pressure.

In accordance with a particularly preferred embodiment of the invention the distance between the means for sensing the pressure to be measured and the blocking balloon is at most five, preferably at most three centimetres. The blocking balloon thus protects the means for sensing the pressure to be measured against being squashed in a fold of the bladder and therefore supplying corrupted measurement results.

In accordance with an advantageous development of the invention the means for sensing the pressure to be measured comprise an in situ pressure transducer which may advantageously be a solid-state pressure transducer. In principle, a large number of pressure sensors which are known per se and are miniaturised or can be miniaturised can be used within the scope of the present invention as in-situ pressure transducers. For example, an optical pressure transducer which operates by the interferometer principle or by means of resilient optical waveguides can also advantageously be used.

Furthermore, a urinary catheter according to the invention can advantageously be equipped with further sensors (advantageously in the region arranged distally from the blocking balloon), for example comprises a temperature sensor (preferably a thermistor) or a chemical sensor for detecting the urea composition.

Fibre optics may also advantageously be provided to carry out tonometry.

In principle, each variant of the invention described or implied within the scope of the present application can be particularly advantageous depending on the economic and technical conditions of the individual case. Unless stated otherwise and insofar as technically feasible in essence, individual features of the embodiments described can be exchanged or combined with one another and with features known per se from the prior art.

The invention will be described hereinafter in greater detail by way of example with reference to drawings. The drawings are not to scale and are purely schematic. In particular, when transferred into practice, the ratios to one another of dimensions of the individually illustrated elements may deviate considerably from the illustration.

A plurality of preferred embodiments will be described, although the invention is not limited thereto.

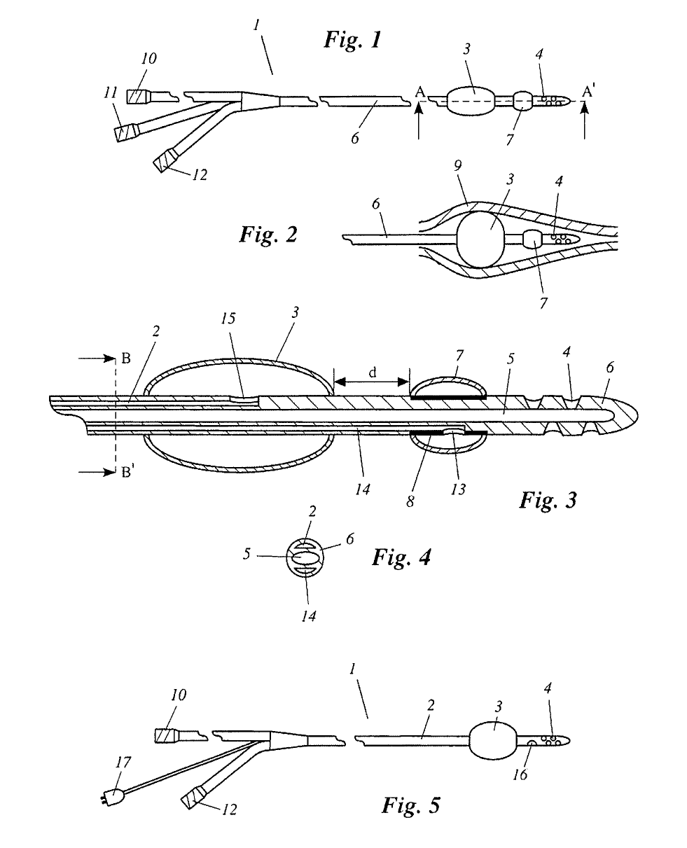

FIG. 1 is a view, with multiple interruptions, of a urinary catheter according to the invention comprising a measurement balloon.

FIG. 2 shows the positioning of a distal portion of the urinary catheter of FIG. 1 in the bladder of patient, the blocking balloon being further inflated compared to FIG. 1.

FIG. 3 shows a longitudinal section through a distal portion of the urinary catheter from FIG. 1, wherein the sectional plane is indicated in FIG. 1 by the dashed line A-A′.

FIG. 4 shows a cross-section through the catheter body of the urinary catheter illustrated in FIG. 1 and FIG. 3, wherein the sectional plane is indicated in FIG. 3 by the dashed line B-B′.

FIG. 5 shows an alternative embodiment of a urinary catheter according to the invention similar to that of FIG. 1, but in this case an in situ pressure transducer is provided instead of the measurement balloon.

The gas-filled measurement balloon 7, which senses bladder pressure in the embodiment illustrated with reference to FIGS. 1 to 4, is arranged on the catheter body 6 distally from the blocking balloon 3. The catheter body 6 is locally reinforced in the region of the measurement balloon 7 by the reinforcement ring 8 in order to avoid effects of a local deformation of the catheter body 6 on the measurement result.

There is a gas connection, via the opening 13 and the measurement lumen 14, between the interior of the measurement balloon 7 and the proximal port 11, to which connection a pressure measurement and evaluation assembly which is known per se can be attached.

Similarly to conventional Foley catheters, the blocking balloon 3 primarily prevents the urinary catheter 1 from being withdrawn too far from the bladder, which is undesirable. It can be inflated with filling gas via the balloon lumen 2 and the opening 15. For this purpose a gas feed device, which is not shown and is known per se from the prior art, can be connected to the proximal port 12, towards which the balloon lumen 2 extends.

Owing to a suitably selected distance d, which is preferably 3 cm or less, between the measurement balloon 7 and the blocking balloon 3, the latter is protected from becoming trapped between a fold in the bladder. FIG. 2 shows how the larger blocking balloon 3 prevents the smaller measurement balloon 7 of the urinary catheter 1 from contacting the bladder wall 9, which would be detrimental to the measurement. In order to remove the urinary catheter 1 the filling gas is discharged from the interior of the blocking balloon 3.

Openings 4 which make it possible to carry off urine through the urine drainage lumen 5 and the proximal port 10, as is the case in conventional Foley catheters, are located distally from the measurement balloon 7.

In the embodiment illustrated in FIG. 5 of a urinary catheter 1 according to the invention the measurement balloon 7 is replaced by an in situ pressure sensor 16. The measurement signal of the pressure sensor 16 can be picked up, recorded and processed via the proximal plug 17 by means of an electronic assembly, of which many variations are known per se from the prior art.

Openings 4 which make it possible to divert urine to the proximal port 10, as is the case in conventional Foley catheters, are located distally from the pressure sensor 16.

Again, the blocking balloon 3 prevents the urinary catheter 1 from being withdrawn too far from the bladder, which is undesirable. For this purpose a gas feed device, which is not shown and is known per se from the prior art, can be connected to the proximal port 12.

1. A urinary catheter for measuring the pressure in the bladder of a living being, comprising:
a catheter body;
a balloon lumen which connects a blocking balloon to a proximal port;
means attached to the catheter body, distally from the blocking balloon, for sensing the pressure to be measured; and
a urine drainage lumen connected to at least one distal opening.

2. The urinary catheter according to claim 1, wherein the means for sensing the pressure to be measured comprises a measurement balloon which is connected to a further proximal port via a measurement lumen.

3. The urinary catheter according to claim 2, wherein the catheter body comprises a reinforcement in the region of the measurement balloon.

4. The urinary catheter according to claim 1, wherein the distance between the means for sensing the pressure to be measured and the blocking balloon is no more than five centimetres.

5. The urinary catheter according to claim 4, wherein the distance between the means for sensing the pressure to be measured and the blocking balloon is no more than three centimetres.

6. The urinary catheter according to claim 1, wherein the means for sensing the pressure to be measured comprises an in situ pressure transducer.

7. The urinary catheter according to claim 6, wherein the in situ pressure transducer is a solid-state pressure transducer.

8. The urinary catheter according to claim 6, wherein the in situ pressure transducer is an optical pressure transducer.

9. The urinary catheter according to claim 1, further comprising a temperature sensor for measuring temperature in the region arranged distally from the blocking balloon.

10. The urinary catheter according to claim 1, further comprising a chemical sensor for measuring and detecting the urea composition.

11. The urinary catheter according to claim 1, further comprising fibre optics for carrying out tonometry.
